(19) United States
(12) Patent Application Publication
(10) Pub. No.: US 2010/0234414 A1
(43) Pub. Date: Sep. 16, 2010

(54) Use of Loratadine 10mg for the prevention of upper respiratory inflammation/sinus infections caused from exposure to bacteria and viruses on airplane flights

(76) Inventor: Mariangela Lazear, Bethesda, MD (US)

Correspondence Address: Mariangela Lazear, 5106 Battery Lane, Bethesda, MD, 20814, US

(21) Appl. No.: 12/404,296

(22) Filed: Mar. 14, 2009

(51) Int. Cl. A61K 31/4545

(52) U.S. Cl. 514/290

(57) ABSTRACT

The invention relates to the use of Loratadine 10 mg prior to flying to prevent upper respiratory inflammation and sinus infections caused from exposure to bacteria and viruses while flying. People are packed together in airplanes, which has dry and polluted air. Flying is one of the more common places to pick up cold viruses and airborne bacteria. Use of Loratadine 10 mg before the plane takes off to shrink the nasal lining helps prevent susceptibility to bacteria and viruses.

### REFERENCES CITED

### U.S. Patent Documents

| | | |
|---|---|---|
| 4,282,233 | August 1981 | Vilani |
| 6,214,837 | April 2001 | Blomqvist |

### Other References

“Sinuses”, The Female Body: An Owner's Manuel, Rodale, www.MotherNature.com

### DESCRIPTION

The present invention relates to the new use of certain compounds known to act as antihistamines for the prevention of upper respiratory inflammation and sinus infections that are frequently caused from exposure to bacteria and viruses while flying on airplanes.

People are packed together in airplanes, which has dry and polluted air. Flying is one of the more common places to pick up cold viruses and airborne bacteria. In accordance with the present invention, the surprising observation has been made that certain substances known to be antihistamines can in certain cases help the prevention of upper respiratory inflammation and sinus infections caused from exposure to bacteria and viruses on airplanes.

Accordingly, taking of Loratadine 10 mg in a tablet form at least 30 minutes before boarding the plane will shrink the nasal lining and will help prevent susceptibility to bacteria and viruses.

Compounds of the general formula and processes for their production are known, for example from U.S. Pat. No. 4,282,233. The compound most preferred for use in the present invention is loratadine (ethyl-4-(8-chloro-5,6-dihydro-11H-benzo(5,6)cyclohepta(1,2b)pyridine-11-ylidene)-1piperidine-carboxylate). This compound is sold by Schering-Plough AB, Stockholm, Sweden in the form of tablets containing 10 mg loratadine, as histamine HI antagonist under the trade name Clarityn, with indications allergic rhinitis and conjunctivitis, and histamine mediated pruritis and urticaria.

What is claimed is:

1. Use of Loratadine 10 mg in tablet form at least 30 minutes before flying will prevent upper respiratory inflammation and sinus infections that are frequently caused from the exposure to bacteria and viruses on airplane flights.

2. The pharmaceutical composition according to claim 1, characterized in that the compound of the general formula is Loratadine 10 mg.

3. A method to prevent susceptibility to bacteria and viruses by orally ingesting Loratadine 10 mg in tablet form 30 minutes before the plane takes off to shrink the nasal lining.

4. The method according to claim 3, wherein said loratadine containing tablet form contains 10 mg of loratadine.
